(19) United States
(12) Patent Application Publication
(10) Pub. No.: US 2026/0102476 A1
(43) Pub. Date: Apr. 16, 2026

(54) USE OF BOTULINUM NEUROTOXIN IN THE TREATMENT OF SIALORRHEA

(71) Applicant: Merz Pharma GmbH & Co. KGaA, Frankfurt am Main (DE)

(72) Inventors: Janos CSIKOS, Frankfurt am Main (DE); Irena PULTE, Eschborn (DE); Michael ALTHAUS, Osten (DE); Markus KRÜER, Kelkheim (DE); Nico WEGENER, Frankfurt am Main (DE)

(21) Appl. No.: 19/368,652

(22) Filed: Oct. 24, 2025

Related U.S. Application Data
(63) Continuation of application No. 18/054,407, filed on Nov. 10, 2022, now Pat. No. 12,478,663.
Application No. 18/054,407 is a continuation of application No. 16/893,799, filed on Jun. 5, 2020, now Pat. No. 11,529,398.
Application No. 16/893,799 is a continuation of application No. 16/129,311, filed on Sep. 12, 2018, now Pat. No. 10,709,772.
Application No. 16/129,311 is a continuation of application No. PCT/EP2018/056850, filed on Mar. 19, 2018.

(30) Foreign Application Priority Data
Mar. 24, 2017 (EP) 17162719.3

(51) Int. Cl. A61K 38/48; A61K 9/00; A61P 1/00; A61P 43/00

(52) U.S. Cl. CPC A61K 38/4893; A61K 9/0019; A61K 9/0053; A61P 1/00; A61P 43/00; Y02A 50/30

(57) ABSTRACT

This invention relates to improved uses of botulinum neurotoxins in the treatment of sialorrhea or diseases or conditions relating to increased saliva production. In particular are botulinum neurotoxins disclosed which are administered into parotid and submandibular glands in a dose ratio between 1.45 to 1 and 1.7 to 1.

### CROSS-REFERENCE TO RELATED APPLICATIONS

This application is a continuation of U.S. patent application Ser. No. 18/054,407, filed Nov. 10, 2022, which is a continuation of U.S. patent application Ser. No. 16/893,799, filed Jun. 5, 2020, now U.S. Pat. No. 11,529,398, issued Dec. 20, 2022, which is a continuation of U.S. patent application Ser. No. 16/129,311, filed Sep. 12, 2018, now U.S. Pat. No. 10,709,772, issued Jul. 14, 2020, which claims priority under 35 U.S.C. § 365 as a continuation of International Application No. PCT/EP2018/056850, filed Mar. 19, 2018, which claims priority to European Patent Application No. 17162719.3, filed Mar. 24, 2017, the entire contents of each of which is incorporated herein by reference.

### REFERENCE TO SEQUENCE LISTING SUBMITTED AS A COMPLIANT XML FILE (.xml)

Pursuant to the EFS-Web legal framework and 37 C.F.R. § 1.821(c), a Sequence Listing is submitted herewith as an XML file named “3000034-005003_Sequence_Listing”, created on Oct. 24, 2025, and having a size of 49,889 bytes. The entire contents of the material in the aforementioned file is hereby incorporated by reference in its entirety.

### BACKGROUND

This invention relates to improved uses of botulinum neurotoxins in the treatment of a disease or condition associated with sialorrhea or increased saliva production. In particular are botulinum neurotoxins disclosed which are administered into parotid and submandibular glands in a dose ratio between 1.45 to 1 and 1.7 to 1.

### BACKGROUND OF THE INVENTION

Drooling is generally represented by a wide variety of clinical conditions which result in the symptom of saliva overflowing the lip margin (known as anterior drooling) or inadvertently overflowing the pharynx, involuntarily entering the glottis and the trachea (known as posterior drooling). As anterior drooling is mainly a problem for patients with regard to social interaction, posterior drooling can also cause cough and irritation in subjects with intact cough reflex or aspired silently in unconscious subjects.

The term drooling is often used in lay language for the medical term sialorrhea, hypersalivation or ptyalism depending on clinical condition, country of usage, specialty of medicine. Per definition, sialorrhea is the “excess spillage of saliva over the lip margin”, hypersalivation is the “excessive production of saliva”, ptyalism is the “hypersalivation in pregnant women”. Those terms and definitions are not consistently used with respect to their unclear cause, and pathomechanism of the underlying conditions and problems.

Causes of sialorrhea can be various and generally relate to an overproduction of saliva or underperformance of saliva managing or eliminating anatomical structures or physiological functions. Of course the combination of those factors makes a clear distinction of causes impossible, therefore the descriptors like diagnoses of the symptoms as listed above are used contradictory. In some cases only anatomical malformations and deformities of salivary glands and ducts, lips, oral cavity, and teeth (defects in lip closure, dental malocclusion) causes local bypasses between the oral cavity and the external world enabling the uncontrolled outflow of the produced saliva.

Malformations, strictures, scars, fistulas and bypasses can occur as permanent consequences of oral or head and neck cancer, injuries and as complications of their surgery. Patients with intellectual disabilities may have permanently open mouth, causing the same effect with or without malformations. Reduced sensorimotor abilities, inefficient oral neuromuscular control, reduced protective reflexes, hypomotility of swallowing muscles, decreased swallowing frequency or ineffective swallowing or dysphagia appear to be the most frequent causes of inherent sialorrhea in patients with neurological conditions selected for example from Parkinson's disease, Progressive Supranuclear Palsy, Corticobasal Degeneration, Multiple System Atrophy, Amyotrophic lateral sclerosis (ALS), cerebral palsy, stroke, traumatic brain injury (TBI), clozapine induced hypersalivation, Rett syndrome, Angelman syndrome, epileptic encephalopathy and brain tumours, total pharyngolaryngectomy, supracricoid laryngectomy and supraglottic laryngectomy, dementia, or intellectual disability or any other cause of sialorrhea or hypersalivation. If not frequently swallowed down due to disturbed movement control of swallowing, the produced unstimulated or stimulated saliva is pooled in the oral cavity. Uncontrolled opening of mouth and anterograde posture of the head facilitates the overflowing of the pooled saliva over the lip margin of the patient.

Salivation can also be increased permanently by irritating factors e.g. massive caries or odontolith, hypertrophy of salivary glands, gastroesophageal reflux or by drugs or toxins inducing hypersalivation as a side effect (e.g. Clozapine, Benzodiazepines, Antipsychotics), causing permanent activation of salivary nuclei or nerve endings in the salivary glands.

Overproduction of saliva can only be controlled in otherwise healthy individuals to a certain extent. In patients with disabled saliva management the upper threshold of the ability to control pooled saliva in the mouth or to swallow the overproduced amount of saliva are lower, therefore more challenging.

Treatment options for swallowing problems focus on rehabilitative measures (swallowing training, oral motor control training) however the unconscious mechanisms of frequent swallowing can hardly be trained and developed in patients with progressive neurological diseases such as Parkinson's disease or ALS. Therefore treatment of sialorrhea is often focused on the reduction of saliva production. Earliest approaches used well-known anticholinergic drugs (e.g. Atropine, Ipratropium Bromide, Scopolamine, Glycopyrrolate, Tropicamide), acting inhibitory on muscarinic cholinergic nerves, which control the amount of produced saliva by salivary glands in and around the oral cavity. Several other derivatives of anticholinergics were also tested and used off label in this indication. Only Glycopyrrolate is approved for the treatment of drooling in children in the USA and EU recently.

Another treatment alternative is botulinum toxin, which is administered to patients by intramuscular injections to reduce muscle tonus and spasticity in treated muscles, or hyperhidrosis. Dry mouth was detected as adverse drug reaction in such patients and this motivated physicians to treat salivary glands with Botulinum toxin A or B directly i.e. by intraglandular or intraparenchymal injections of Botulinum toxin A or B into the major salivary glands parotids and submandibular glands.

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TeNT) function via a similar initial physiological mechanism of action, inhibiting neurotransmitter release from the axon of the affected neuron into the synapse, they differ in their clinical response. While the botulinum neurotoxin acts at the neuromuscular junction and other cholinergic synapses in the peripheral nervous system, inhibiting the release of the neurotransmitter acetylcholine and thereby causing flaccid paralysis, the tetanus neurotoxin, which is transcytosed into central neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/H with further subtypes. Most Clostridium botulinum strains produce one type of neurotoxin, but strains producing multiple neurotoxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial neurotoxins, is located between the two cysteine residues forming the disulfide bond. This loop region is proteolytically cleaved by an unknown clostridial endoprotease to obtain the biologically active neurotoxin.

The molecular mechanism of intoxication by TeNT and BONT appears to be similar as well: entry into the target neuron is mediated by binding of the C-terminal part of the heavy chain to a specific cell surface receptor; the neurotoxin is then taken up by receptor-mediated endocytosis. The low pH in the so formed endosome then triggers a conformational change in the clostridial neurotoxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TeNT, BoNT/B, BONT/D, BONT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C1 cleaves the integral plasma membrane protein syntaxin and SNAP-25.

In Clostridium botulinum, the botulinum neurotoxin is formed as a protein complex comprising the neurotoxic component and non-toxic proteins. The accessory proteins embed the neurotoxic component thereby protecting it from degradation by digestive enzymes in the gastrointestinal tract without adding anything to the toxic effect. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as the treatment of fine wrinkles. Preparations comprising botulinum neurotoxin complexes are commercially available, e.g. from Ipsen Ltd (Dysport®), Solstice Neurosciences LLC/US Worldmeds LLC (Myobloc®) or Allergan Inc. (Botox®). A high purity neurotoxic component of botulinum neurotoxin, free of any complexing proteins, is for example available from Merz Pharmaceuticals GmbH, Frankfurt (Xeomin®, Bocouture®).

There are a couple of reports in the prior art about the use of botulinum neurotoxin A and B in patients with sialorrhea caused by different underlying diseases. For example, Breheret et al. (Annales francaises d Óto-rhino-laryngologie et de Pathologie Cervico-faciale, volume 128, Issue 5, 2011, pages 266-271), Barbero et al. (J Neurol. 2015 December; 262 (12): 2662-7), Suskind et al. (Laryngoscope. 2002 January; 112 (1): 73-81), Porta et al. (J Neurol Neurosurg Psychiatry. 2001 April; 70 (4): 538-40.), Narayanaswami et al. (Parkinsonism Relat Disord. 2016 September; 30:73-7) and Castelnovo et al. (Movement Disorders 2013, Volume 28, Abstract Supplement) report the use of different toxins according to several different protocols with varying amounts of toxin administered to the salivary glands for treating sialorrhea in a variety of medical conditions. Despite the number of studies providing data about safety and efficacy of using botulinum neurotoxins in sialorrhea, there are still a lot of ongoing discussions without clear recommendations about the dosages, the sites of administration and the type of toxin to be used.

### BRIEF SUMMARY

One of the objects of the present invention is to provide a botulinum neurotoxin for treatment of a disease or condition associated with sialorrhea or increased saliva production which limits the activity of the salivary glands for a long period of time with an extent that the subject shows no drooling but which still allows that the reduced amount of produced saliva is sufficient for normal physiologic functioning, e.g. as lubricant, as ion reservoir, as buffer, as cleansing, for antimicrobial actions, for agglutination, for pellicle formation, for digestion, for taste, for excretion and/or for water balance. Another object of the present invention is to avoid side effects related to the treatment with a botulinum neurotoxin or to reduce them at least in frequency, severity and/or duration while under treatment. As a further object of the present invention, the botulinum neurotoxins should provide advantageous treatment results over a long period of treatment as the underlying disease will not be affected by the treatment, therefore long lasting therapy should be applicable effective and safe without waning of efficacy or compromising safety with repeated injection cycles for prolonged treatment.

Surprisingly, it has been identified that a botulinum neurotoxin can address one or more of these objections, if it is used in treating a disease or condition associated with sialorrhea or increased saliva production, wherein said botulinum neurotoxin is administered by injection into parotid glands and submandibular glands and wherein the ratio between the doses of botulinum neurotoxin administered to each of the parotid glands and each of the submandibular glands is between 1.45 to 1 and 1.7 to 1.

### BRIEF DESCRIPTION OF THE SEVERAL VIEWS OF THE DRAWINGS

FIG. 1 shows mean reduction in objectively measured unstimulated salivary flow rate (uSFR) from baseline (FAS).

FIG. 2 shows mean global impression of change scale (GICS) (FAS). Superiority of NT 201 over placebo is shown by mean Global Impression of Change Scale (GICS) [FAS].

FIG. 3 shows response rate in subject's GICS over time (FAS).

FIG. 4 shows mean reduction of drooling severity and frequency sum score (DSFS) from baseline (FAS). Superiority of NT 201 over placebo is shown by the mean reduction of drooling severity and frequency sum score (DSFS) from baseline [FAS].

FIG. 5 shows mean mROMP drooling subscore reduction from baseline (FAS).

### DETAILED DESCRIPTION OF THE INVENTION

The present invention may be understood more readily by reference to the following detailed description of the invention and the examples included therein.

In a first embodiment the present invention relates to a botulinum neurotoxin for use in treating a disease or condition associated with sialorrhea or increased saliva production, wherein said botulinum neurotoxin is administered by injection into parotid glands and submandibular glands and wherein the ratio between the doses of botulinum neurotoxin administered to each of the parotid glands and each of the submandibular glands is between 1.45 to 1 and 1.7 to 1. In a preferred embodiment the botulinum neurotoxin of the present invention is administered into parotid glands and submandibular glands wherein the ratio between the doses of botulinum neurotoxin administered to each of the parotid glands and each of the submandibular glands is between 1.50 to 1 and 1.6 to 1. In a particular preferred embodiment the botulinum neurotoxin of the present invention is administered into parotid glands and submandibular glands wherein the ratio between the doses of botulinum neurotoxin administered to each of the parotid glands and each of the submandibular glands is 1.50 to 1.

In a further embodiment the present invention relates to a method of treating a disease or condition associated with sialorrhea or increased saliva production in a patient, the method comprising administering a therapeutically effective amount of a botulinum neurotoxin by injection into parotid glands and submandibular glands, wherein the ratio between the doses of botulinum neurotoxin administered to each of the parotid glands and each of the submandibular glands is between 1.45 to 1 and 1.7 to 1. In a preferred embodiment the method of the present invention comprises administering a therapeutically effective amount of a botulinum neurotoxin by injection into parotid glands and submandibular glands, wherein the ratio between the doses of botulinum neurotoxin administered to each of the parotid glands and each of the submandibular glands is between 1.50 to 1 and 1.6 to 1. In a particular preferred embodiment the method of the present invention comprises administering a therapeutically effective amount of a botulinum neurotoxin by injection into parotid glands and submandibular glands, wherein the ratio between the doses of botulinum neurotoxin administered to each of the parotid glands and each of the submandibular glands is 1.50 to 1.

In a further aspect the present invention generally relates to botulinum toxins, for treating a disease or condition associated with sialorrhea or increased saliva production. In particular embodiments of the present invention the disease or condition associated with sialorrhea or increased saliva production is associated for example with Parkinson's disease, Progressive Supranuclear Palsy, Corticobasal Degeneration, Multiple System Atrophy, Amyotrophic lateral sclerosis (ALS), cerebral palsy, stroke, traumatic brain injury (TBI), clozapine induced hypersalivation, Rett syndrome, Angelman syndrome, epileptic encephalopathy, brain tumours, total pharyngolaryngectomy, supracricoid laryngectomy and supraglottic laryngectomy, dementia, or intellectual disability (e.g. global developmental delay, severe learning disability) or any other cause of sialorrhea or hypersalivation. A disease or condition associated with sialorrhea or increased saliva production according to the present invention can be also Down's syndrome, Smith-Lemli-Opitz syndrome, Möbius syndrome, MEGDEL syndrome, Beckwith-Wiedemann syndrome, lymphatic malformation of the tongue, Foix-Chavany-Marie syndrome, chromosomal abnormalities and genetic diseases like 17q21 deletion, familial dysautonomia, partial trisomy 22, Aicardi syndrome, SMA Type 1, GMI gangliosidosis or Apert syndrome, Wilson disease, congenital brain malformation like hydrocephalus, microcephaly, pontocerebellar hypoplasia, posterior fossa mass, neuronal ceroid lipofuscinosis, Batten disease, metachromatic leukodystrophy, multiplex arthrogryposis, encephalopathy, lissencephaly or pachigyria, brain injuries like spinal cord injury, hypoxic ischemic encephalopathy, congenital toxoplasmosis, congenital CMV infection, post meningoencephalitis or post herpes encephalitis, neuromotor disorders like oral dyspraxia, suprabulbar palsy, operculum syndrome, myopathy, infantile spasms, myotonic dystrophy, Duchenne muscular dystrophy, Neurofibromatosis type I or mitochondriopathy, fetal alcohol syndrome, autism or juvenile Guillain-Barré Syndrome.

In particular embodiments of the present invention the disease or condition associated with sialorrhea or increased saliva production is associated with stroke, in particular the disease or condition associated with sialorrhea or increased saliva production occurred after stroke (post stroke).

In preferred embodiments of the present invention the disease or condition associated with sialorrhea or increased saliva production is associated for example with traumatic brain injury (TBI), post stroke, Parkinson's disease or atypical parkinsonism (Progressive Supranuclear Palsy [PSP], Multisystem Atrophy [MSA], Corticobasal Degeneration [CBD]). In another preferred embodiment of the present invention the disease or condition associated with sialorrhea or increased saliva production is traumatic brain injury (TBI), post stoke, Parkinson's disease or atypical parkinsonism (Progressive Supranuclear Palsy [PSP], Multisystem Atrophy [MSA], Corticobasal Degeneration [CBD]) with chronic sialorrhea for at least 3 months duration and sialorrhea severity of at least 2 score points on the Drooling Severity Subscale and a frequency of at least 2 score points on the Drooling Frequency Subscale and at least 6 score points on the sum score Drooling Severity and Frequency Scale. In another preferred embodiment of the present invention the disease or condition associated with sialorrhea or increased saliva production is traumatic brain injury (TBI), post stoke, Parkinson's disease or atypical parkinsonism (Progressive Supranuclear Palsy PSP, Multisystem Atrophy MSA, Corticobasal Degeneration CBD) with chronic sialorrhea having at least 0.3 g/min unstimulated salivary flow rate.

The present invention relates in a further embodiment to a pharmaceutical composition comprising a botulinum neurotoxin for the use in treating a disease or condition associated with sialorrhea or increased saliva production, and a pharmaceutical acceptable carrier, wherein said botulinum neurotoxin is administered by injection into parotid glands and submandibular glands and wherein the ratio between the doses of botulinum neurotoxin administered to each of the parotid glands and each of the submandibular glands is between 1.45 to 1 and 1.7 to 1.

According to one embodiment of the present invention the botulinum neurotoxin is administered into parotid glands and submandibular glands in a total dose between 70 U and 110 U. In a preferred embodiment the total dose of botulinum neurotoxin administered into parotid and submandibular glands is between 75 U and 100 U.

According to one embodiment of the present invention the botulinum neurotoxin is administered into parotid glands and submandibular glands in a total dose of 75 U. In an alternative embodiment the total dose of botulinum neurotoxin administered into parotid and submandibular glands is 100 U.

Generally, the botulinum neurotoxin can be administered according to the present invention into parotid glands and submandibular glands in a total dose between 0.5 and 2.35 U/Kg body weight. In a particular preferred embodiment the botulinum neurotoxin is administered into parotid glands and submandibular glands in a total dose between 1 and 1.25 U/Kg body weight. Due to the low body weight botulinum toxin is generally administered in children as displayed in the dosing table 8. In another embodiment total dosage of up to 2.5 U/kg are administered into parotid and submandibular gland in children.

According to a further aspect of the present invention, the botulinum neurotoxin is administered in an aqueous composition having a botulinum neurotoxin concentration in the range between 45 and 55 U/mL. In a preferred embodiment of the present invention the botulinum neurotoxin is administered as aqueous composition having a botulinum neurotoxin concentration of 50 U/mL. In a particular preferred embodiment the contents of a 100 U vial will be reconstituted with a total of 2.0 mL physiological saline and the volumes administered to parotid and submandibular glands are:
Parotid gland: 0.6 ml on each side,
Submandibular gland: 0.4 ml on each side.

If several consecutive treatment cycles are envisaged the injection volumes can be reduced if dry mouth or dysphagia occurs at previous treatment cycles. This reduction is recommended at the discretion of the injector to avoid further occurrence of such side effects. If the administration of reduced botulinum neurotoxin quantities is envisaged, the injection volumes administered to parotid and submandibular glands are:
Parotid gland: 0.45 ml on each side,
Submandibular gland: 0.3 ml on each side.

The biological activity is commonly expressed in Mouse Units (U). As used herein, 1 U is the amount of neurotoxic component of the botulinum neurotoxin, which kills 50% of a specified mouse population after intraperitoneal injection, i.e. the mouse i.p. LD50. Another particular useful method for determining the biological activity of a botulinum neurotoxin is a cell-based assay as it is disclosed for example in WO2009/114748, WO 2013/049508 or WO 2014/207109. The activity results obtained with such cell-based assays correspond to the activity values obtained in the mouse i.p. LD50 assay. Activity results obtained for Botulinum serotype A formulations like commercially available Incobotulinumtoxin A (Botulinumtoxin serotype A, without complexing proteins, Xeomin®, Merz Pharmaceuticals GmbH)) or Onabotulinumtoxin A (Botulinumtoxin serotype A, with complexing proteins, Botox®, Allergan Inc.) can be converted to values for other toxins using conversion rates known to the person skilled in the art. For example, the necessary dose of AbobotulinumtoxinA A (Botulinumtoxin serotype A, with complexing proteins, Dysport®, Ipsen Biopharm Limited) can be determined by multiplication of the dose of Incobotulinumtoxin A or Onabotulinumtoxin A with a factor of 2.5 to 5. The dose for RimabotulinumtoxinB (Botulinumtoxin serotype B, Myobloc®, Solstice Neurosciences/US WorldMeds LLC) can be calculated by multiplication of the dose of Incobotulinumtoxin A or Onabotulinumtoxin A with a factor of 20 to 40.

In a further embodiment of the present invention the botulinum neurotoxin is administered in a volume of between 0.3 and 0.5 mL per injection site into the submandibular glands and in a volume of between 0.5 to 0.7 mL per injection site into the parotid glands. In a particular preferred embodiment of the present invention the botulinum neurotoxin is administered in a volume of 0.4 mL per injection site into the submandibular glands and in a volume of 0.6 mL per injection site into the parotid glands.

In a further embodiment of the present invention the botulinum neurotoxin is injected into one site of each submandibular gland on both sides of the patient. Injections are applied into the geometrically centrum of the glands, depending on the anatomical extent of the gland.

In another embodiment of the present invention the botulinum neurotoxin is injected into one site of each parotid gland on both sides of the patient. Injections are applied into the geometrical centrum of the glands, depending on the anatomical extent of the gland.

In a preferred embodiment the total dose of botulinum neurotoxin is injected into one site of each submandibular gland and into one site of each parotid gland.

One embodiment of the present invention relates to a botulinum neurotoxin for use in treating a disease or condition associated with sialorrhea or increased saliva production, wherein said botulinum neurotoxin is administered by injection into parotid glands and submandibular glands and wherein the ratio between the doses of botulinum neurotoxin administered into each of the parotid glands and each of the submandibular glands is between 1.45 to 1 and 1.7 to 1, wherein the disease or condition associated with sialorrhea or increased saliva production is associated with stroke and wherein a total dose of 100 U of the botulinum neurotoxin is injected into one site of each submandibular gland and into one site of each parotid gland.

One embodiment of the present invention relates to a botulinum neurotoxin for use in treating a disease or condition associated with sialorrhea or increased saliva production, wherein said botulinum neurotoxin is administered by injection into parotid glands and submandibular glands and wherein the ratio between the doses of botulinum neurotoxin administered into each of the parotid glands and each of the submandibular glands is 1.5 to 1, wherein the disease or condition associated with sialorrhea or increased saliva production is associated with stroke and wherein a total dose of 100 U of the botulinum neurotoxin is injected into one site of each submandibular gland and into one site of each parotid gland, in particular into the geometrical centrum of the gland, respectively.

In particular embodiments of the present invention the botulinum neurotoxin is injected into parotid glands and submandibular glands without using ultrasound guidance. In this case the target site within the gland is determined by using anatomical landmark orientation as it is well known for a person skilled in the art. The parotid gland is located inferior and anterior to the external acoustic meatus and lies posterior to the mandibular ramus and anterior to the mastoid process of the temporal bone. The gland is roughly wedge shaped when seen superficially and is also wedge shaped when seen on horizontal sections. The parotid gland can be easily palpated. To find palpable landmarks for the parotid gland one should palpate between the mandibular anterior ramus and the sternocleidomastoideole. Starting palpating anterior to each ear, moving to the cheek area, and then inferior to the angle of the mandible. Using the anatomic landmarks the superficial borders of the parotid gland are palpated and the botulinum neurotoxin is injected into the middle of the parotid gland. Injection can be given into the upper or lower halves of the main glandular body. A single injection point needs to be selected. The same procedure applies to the other side of the subject. The submandibular gland is located beneath the floor of the mouth below the mandibular arch next to the following anatomic structures. Lying superior to the digastric muscles, each submandibular gland is divided into superficial and deep lobes, which are separated by the mylohyoid muscle. The superficial lobe comprises most of the gland, with the mylohyoid muscle runs under it. The deep lobe is the smaller part. Although the submandibular gland is not always easily palpable, its anatomical position is well defined. The injection is given, albeit very rarely, parallel to the excretory duct. The submandibular gland will be injected by fixating the gland with two fingers in the position below the mandibula. The needle will be inserted from the upwards forwards in the direction of the mouth floor in 70-90 degree to the mandibula (Holsinger 2005, Anatomy, Function, and Evaluation of the Salivary Glands).

In other embodiments the botulinum neurotoxin is injected into parotid glands and submandibular glands using ultrasound guidance. A person skilled in the art is well aware of applying ultrasound imaging techniques to fully determine size and localization of the target area within the body of the gland. A high frequency linear transducer >7.5 MHz can be used, for example, to identify and visualize the gland [Howlett, High resolution ultrasound assessment of the parotid gland (2003) British Journal of Radiology 76, 271-277].

It is generally envisaged that the botulinum neurotoxin is injected into parotid glands and submandibular glands more than one time. In particular embodiments the botulinum neurotoxin according to the present invention is administered in consecutive treatment cycles. According to the present invention a treatment cycle is the time interval between two administrations of the botulinum neurotoxin, i.e. a treatment cycle consists of one administration of the botulinum neurotoxin and a follow-up period until the next botulinum neurotoxin injection is administered. The botulinum neurotoxin is preferably administered in at least 2, at least 3, at least 4, at least 5, at least 6, at least 7 or at least 8 treatment cycles. In one embodiment the botulinum neurotoxin is administered in 2 to 6 treatment cycles, in particular in 4 treatment cycles.

The time interval between two consecutive administrations of the botulinum neurotoxin into parotid glands and submandibular glands can vary between 10 and 20 weeks or between 12 and 20 weeks. In another embodiment the time interval between two consecutive administrations of the botulinum neurotoxin into parotid glands and submandibular glands can vary between 6 and 10 weeks. In a preferred embodiment the time interval between two consecutive administrations the botulinum neurotoxin into parotid glands and submandibular glands vary between 12 and 18 weeks, or between 14 and 18 weeks. In a most preferred embodiment the time interval is 15, 16 or 17 weeks, in particular 16 weeks.

In one embodiment of the invention the time interval remains the same between all consecutive administrations of the botulinum neurotoxin into parotid glands and submandibular glands.

In one embodiment of the present invention the botulinum neurotoxin is injected into parotid glands and submandibular glands in at least 4 consecutive treatment cycles, wherein the time interval between the consecutive administrations of the botulinum neurotoxin is 16 weeks.

Generally, there are several ways to determine the efficacy of a botulinum toxin for the treatment of a disease or condition associated with sialorrhea or increased saliva production known to the person skilled in the art. Measurements and scales for determining the efficacy of a botulinum toxin for the treatment of sialorrhea or a disease or condition associated with increased saliva production can be selected from e.g. determining unstimulated Salivary Flow Rate (uSFR), Mean Global Impression of Change Scale (GICS), Drooling Severity and Frequency Scale (DSFS), modified Radboud Oral Motor Inventory for Parkinson's Disease (mROMP), Modified Teacher's Drooling Scale (mTDS), Drooling Impact Scale (DIS), Drooling Quotient (DQ) Drooling Rating Scale (DRS) and/or UPDRS Drooling Scale.

In particular embodiments at least two of these measurements and scales can be combined for determining the efficacy of a botulinum toxin for the treatment of sialorrhea or a disease or condition associated with increased saliva production.

In one embodiment of the present invention the botulinum toxin for the treatment of sialorrhea or a disease or condition associated with increased saliva production is used in a patient having a baseline saliva production, i.e. unstimulated Salivary Flow Rate (uSFR) between 0.1-1.6 g/min. In a preferred embodiment the botulinum toxin for the treatment of sialorrhea or a disease or condition associated with increased saliva production is used in a patient having a baseline saliva production, i.e. unstimulated Salivary Flow Rate (uSFR) of more than 0.3 g/min. In another embodiment the botulinum toxin for the treatment of sialorrhea or a disease or condition associated with increased saliva production is used in a patient having at baseline a Drooling Severity and Frequency Scale (DSFS) Sum Score ≥6 and Severity Subscore ≥2 and Frequency Subscore ≥2. Generally the determination of the uSFR and DSFS scores is well known to a person skilled in the art. According to the present invention the uSFR is determined by the weight of collected saliva for 5 minutes using four absorptive swabs for collection. Collection of saliva is performed by placing adsorptive material into the oral cavity (e.g. four dental rolls, Salivette® or Salimetrics Oral Swabs®) for 5 minutes. The absorptive material adsorbs saliva from the closed oral cavity and weight gain of absorptive material due to the collected amount of saliva can be determined by measuring the weight of the absorptive material before and after placing it into the oral cavity. A repetition of the collection and measurement of the amount of produced saliva for 5 minutes after a pause of 30 minutes provides a second value. The average of both values guarantees the reliability of measurement results (by reducing intraindividual variability of measurements) (Jongerius P H, van Limbeek J, Rotteveel J J. Assessment of salivary flow rate: biologic variation and measure error. Laryngoscope. 2004; 114 (10): 1801-4).

In a further embodiment of the present invention the administration of 100 U botulinum neurotoxin reduces the uSFR by at least 25% compared to baseline within 4 weeks after administration. In a preferred embodiment the administration of 100 U botulinum neurotoxin reduces the uSFR by at least 30% (median) compared to baseline within 4 weeks after injection. In another embodiment of the present invention the administration of 100 U botulinum neurotoxin reduces the uSFR by at least 22% (median) compared to baseline within 8 weeks after administration. In a preferred embodiment the administration of 100 U botulinum neurotoxin reduces the uSFR by at least 28% (median) compared to baseline within 8 weeks after injection.

In a further embodiment of the present invention the administration of 100 U of the botulinum neurotoxin improves the Global Impression of Change Scale (GICS) score for drooling assessed by the patient by at least +0.90 score points on a 7 point Likert like scale compared to baseline drooling within 4 weeks after administration. In a preferred embodiment the administration of 100 U of the botulinum neurotoxin shows a Global Impression of Change Scale (GICS) improvement of at least +1.00 score points compared to baseline drooling within 4 weeks after injection. In another embodiment of the present invention the administration of 100 U of the botulinum neurotoxin improves drooling measured by a Global Impression of Change Scale (GICS) by at least +1.00 score points compared to baseline drooling within 8 weeks after administration. In a preferred embodiment the administration of 100 U of the botulinum neurotoxin improves drooling measured by the Global Impression of Change Scale (GICS) by at least +0.90 score points compared to baseline within 12 weeks after injection. The Global Impression of Change Scale (GICS) is determined by a Likert-like scale answering the question “Compared to how you were doing just before the last injection into your salivary gland, what is your overall impression of how you are functioning now as a result of this treatment?” with scale answers ranging from “−3 very much worse” to “+3 very much improved” (Likert, Rensis (1932). “A Technique for the Measurement of Attitudes”. Archives of Psychology. 140:1-55)).

In a further embodiment of the present invention the administration of 100 U of the botulinum neurotoxin reduces the mean Drooling Severity and Frequency Scale (DSFS) sum score by at least 0.90 score points compared to baseline within 4 weeks after administration. In a preferred embodiment the administration of 100 U of the botulinum neurotoxin reduces the mean Drooling Severity and Frequency Scale (DSFS) sum score by at least 1.20 score points compared to baseline within 4 weeks after injection. In another embodiment of the present invention the administration of 100 U of the botulinum neurotoxin reduces the mean Drooling Severity and Frequency Scale (DSFS) sum score by at least 1.50 score points compared to baseline within 8 weeks after administration. The Drooling Severity and Frequency Scale (DSFS) is determined by two subscales, a 4-point Likert scale for ‘drooling frequency’ and a 5-point Likert scale for ‘drooling severity’. The DSFS is the sumscore of the two subscales. The evaluation refers to the time period, “over the past week”. The highest possible score is 9 (Thomas-Stonell N, Greenberg J. Three treatment approaches and clinical factors in the reduction of drooling. Dysphagia. 1988; 3(2): 73-8.).

### Drooling Severity

1 Dry (never drools)
2 Mild (only lips wet)
3 Moderate (wet on lips and chin)
4 Severe (drool extends to clothes wet)
5 Profuse (hands, tray and objects wet)

### Drooling Frequency

1 Never
2 Occasionally (not every day)
3 Frequently (part of everyday)
4 Constantly

In a further embodiment of the present invention the administration of 100 U of the botulinum neurotoxin reduces the mean modified Radboud Oral Motor Inventory for Parkinson's Disease (mROMP) Saliva Control Domain sum score by at least 3.50 score points compared to baseline within 4 weeks after administration. In a preferred embodiment the administration of 100 U of the botulinum neurotoxin reduces the mean modified Radboud Oral Motor Inventory for Parkinson's Disease (mROMP) Saliva Control Domain sum score by at least 4.60 score points compared to baseline within 4 weeks after injection. In another embodiment of the present invention the administration of 100 U of the botulinum neurotoxin reduces the modified Radboud Oral Motor Inventory for Parkinson's Disease (mROMP) Saliva Control Domain sum score by at least 5.5 score points compared to baseline within 8 weeks after administration. In a preferred embodiment the administration of 100 U of the botulinum neurotoxin reduces the modified Radboud Oral Motor Inventory for Parkinson's Disease (mROMP) Saliva Control Domain sum score by at least 6.50 score points compared to baseline within 8 weeks after injection. The modified Radboud Oral Motor Inventory for Parkinson's Disease (mROMP) is determined by original ROMP Inventory [Kalf 2011, Arch. Phys. Med. Rehabil.] which is a Dutch 23-item questionnaire of 5-point Likert scales in the domains speech, swallowing and saliva control. The ROMP was modified (mROMP) to implement small changes in wording resulting from patient interviews during linguistic validation into US English. The mROMP has now 24 items with clearly distinguishable response options and a recall period of the last 7 days.

In one aspect of the present invention the botulinum neurotoxin is a botulinum neurotoxin complex. Complexes with, for example, 450 kDa or with 900 kDa are obtainable from cultures of Clostridium botulinum. A Clostridium botulinum neurotoxin complex according to the present invention comprises the neurotoxic component and non-toxic proteins. The accessory proteins embed the neurotoxic component thereby protecting it from degradation by digestive enzymes in the gastrointestinal tract without adding anything to the toxic effect.

In another aspect of the present invention the botulinum neurotoxin is the neurotoxic component of a botulinum neurotoxin complex. Generally the neurotoxic component has a molecular weight of 150 kDa. The neurotoxic component is devoid of any other protein component of the Clostridium botulinum neurotoxin complex.

The botulinum neurotoxin according to the present invention is selected from the group of different serotypes including botulinum neurotoxin serotype A (BoNT/A), botulinum neurotoxin serotype B (BoNT/B), botulinum neurotoxin serotype C1 (BoNT/C1), botulinum neurotoxin serotype D (BoNT/D), botulinum neurotoxin serotype E (BoNT/E), botulinum neurotoxin serotype F (BoNT/F) or botulinum neurotoxin serotype G (BoNT/G). The botulinum neurotoxin and, in particular, its light chain and heavy chain are derivable from one of the antigenically different serotypes of botulinum neurotoxins indicated above. In an aspect, said light and heavy chain of the botulinum neurotoxin are the light and heavy chain of a botulinum neurotoxin selected from the group consisting of: BONT/A, BoNT/B, BONT/C1, BONT/D, BONT/E, BoNT/F, or BoNT/G. In another aspect, a polynucleotide encoding said botulinum neurotoxin of the present invention comprises a nucleic acid sequence as shown in SEQ ID NO: 1 (BoNT/A), SEQ ID NO: 3 (BoNT/B), SEQ ID NO: 5 (BoNT/C1), SEQ ID NO: 7 (BoNT/D), SEQ ID NO: 9 (BoNT/E), SEQ ID NO: 11 (BoNT/F), or SEQ ID NO: 13 (BoNT/G). Moreover, encompassed is, in an aspect, a polynucleotide comprising a nucleic acid sequence encoding an amino acid sequence as shown in any one of SEQ ID NO: 2 (BONT/A), SEQ ID NO: 4 (BONT/B), SEQ ID NO: 6 (BoNT/C1), SEQ ID NO: 8 (BoNT/D), SEQ ID NO: 10 (BONT/E), SEQ ID NO: 12 (BoNT/F), or SEQ ID NO: 14 (BoNT/G). Further encompassed is in an aspect of the means and methods of the present invention, a botulinum neurotoxin comprising or consisting of an amino acid sequence selected from the group consisting of: SEQ ID NO: 2 (BONT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), and SEQ ID NO: 14 (BoNT/G).

In another aspect, the said polynucleotide encoding a botulinum neurotoxin of the present invention is a variant of the aforementioned polynucleotides comprising one or more nucleotide substitutions, deletions and/or additions which in still another aspect may result in a polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 96%, at least 97%, at least 98% or at least 99% identical to the nucleic acid sequence as shown in any one of SEQ ID NOs: 1, 3, 5, 7, 9, 11 or 13 or a nucleic acid sequence variant which encodes an amino acid sequence being at least 40%, at least 50%, at least 60%, at least 70%, at least 75%, at least 80%, at least 85%, at least 90%, at least 95%, at least 96%, at least 97%, at least 98% or at least 99% identical to the amino acid sequence as shown in any one of SEQ ID NOs: 2, 4, 6, 8, 10, 12, or 14. The term “identical” as used herein refers to sequence identity characterized by determining the number of identical amino acids between two nucleic acid sequences or two amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wisconsin, USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a polypeptide retaining one or more and, in another aspect, all of the biological properties of the respective botulinum neurotoxin, i.e. the BoNT/A, BoNT/B, BONT/C1, BONT/D, BONT/E, BoNT/F or BoNT/G. Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In vivo assays for assessing biological activity include the mouse LD50 assay and the ex vivo mouse hemidiaphragm assay as described by Pearce et al. (Pearce 1994, Toxicol. Appl. Pharmacol. 128:69-77) and Dressler et al. (Dressler 2005, Mov. Disord. 20:1617-1619, Keller 2006, Neuroscience 139:629-637) or a cell-based assay as described in WO2009/114748, WO2014/207109 or WO 2013/049508. The biological activity is commonly expressed in Mouse Units (U). As used herein, 1 U is the amount of neurotoxic component of the botulinum neurotoxin, which kills 50% of a specified mouse population after intraperitoneal injection, i.e. the mouse i.p. LD50. In a further aspect, the variant polynucleotides can encode botulinum neurotoxins having improved or altered biological properties, e.g., they may comprise cleavage sites which are improved for enzyme recognition or may be improved for receptor binding or any other property specified above. A particular useful method for determining the biological activity of a botulinum neurotoxin is a cell-based assay as it is disclosed for example in WO2009/114748, WO 2013/049508 or WO 2014/207109.

Without being bound to theory it is furthermore envisaged that in particular a formulation of a botulinum neurotoxin free of complexing proteins (incobotulinumtoxin A Xeomin®), i.e. the neurotoxic component of botulinum neurotoxin being devoid of any other protein component of the Clostridium botulinum neurotoxin complex, in comparison to other botulinum neurotoxins with complexing proteins (Onabotulinumtoxin A, Botox®, AbobotulinumtoxinA, Dysport®, RimabotulinumtoxinB, Myobloc® or others with complexing proteins), allows a clinically reversible, functional inactivation of the cholinergic neural transmission without disrupting the intracellular structure of the salivary glands and salivary ducts. The use of the neurotoxic component of botulinum neurotoxin being devoid of any other protein component of the Clostridium botulinum neurotoxin complex also does not cause any physical damage in acinar cells as described in resected submandibular salivary glands of children after Botulinum toxin injections [Mosseri 2016, Otolaryngology-Head and Neck Surgery].

Generally, the blockade of nerve terminals by botulinum neurotoxins is irreversible; the clinical effects, however, are temporary as new nerve terminals sprout giving rise to new connections. Complexing proteins are regarded as biologically inactive compounds for treatment and they are generally considered to play no role in the efficacy of botulinum neurotoxins used in intramuscular injections for the treatment of spasticity, dystonia, hyperhidrosis, headache, depression, urinary detrusor spasm or in aesthetic indications like glabellar frown lines or wrinkles.

Complexing proteins are remnants of Clostridial proteins, which originate from the bacteria Clostridium botulinum. Those proteins are produced together with the neurotoxic component of the botulinum neurotoxin protein complex and they play a fundamental role in protection of the toxin in aggressive environments (e.g. acidic conditions in the stomach) and they help the internalization of the toxin through the epithelial barrier of the intestines. Complexing proteins consist of hemagglutinins and non-hemagglutinins and are considered as non-toxic proteins of the botulinum toxin protein complex. Hemagglutinins (HA) were described to disrupt the intercellular epithelial barrier in intestines by directly binding E-cadherin [Fujinaga 2009, Toxicon], [Sugawara et al 2010 J. Cell Biol.], [Lee 2014, Science]. In salivary glands secretory epithelium, intercalated ductal epithelium and striated ductal epithelium develop from ectodermal germ lines similar to the intestinal epithelium. Of particular interest in the tight junctions of the salivary glands are the members of cadherin family, which play a role in salivary gland development, tissue organization, and cell differentiation. Epithelial (E)-cadherin is the main cell-cell adhesion molecule in epithelial tissues and is regarded as a master organizer of the epithelial phenotype. [Davies 2006, Developmental Cell]. In early morphogenesis, E-cadherin and β-catenin are likely to participate in salivary gland remodelling, whereas during cytodifferentiation, they form stable cell-cell contacts and may collaborate with Rho GTPases in the establishment and maintenance of salivary cell polarity” [Baker 2010, Journal of Biomedicine and Biotechnology. The unique intercellular structures like E-cadherins play a fundamental role in polarization of epithelial cells in intestines and salivary glands as well. Interference of E-cadherins with the complexing proteins of Botulinum toxins therefore interferes with the normal biological activity of the salivary glands. Xu and Shan, for example, demonstrated that after BoNT/A Prosigne® Hengli® (Lanzhou Biochemical Co., Gansu, China administration (i.e. a botulinum neurotoxin with complexing proteins) into rat submandibular glands, Aquaporin (AQP5) on the glandular cell membrane is downregulated, which may be a secondary effect of denervation (Xu et al. 2015 Journal of Dental Research, Shan et al. 2013 International Journal of Oral Science).

For preparing a pharmaceutical preparation comprising a botulinum neurotoxin the neurotoxin can be formulated by various techniques dependent on the desired application purposes which are known in the art. For example, the (biologically active) botulinum neurotoxin can be used in combination with one or more pharmaceutically acceptable carriers as a pharmaceutical composition. The pharmaceutically acceptable carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatine, agar, pectin, acacia, magnesium stearate, stearic acid and the like. Exemplary of liquid carriers are glycerol, phosphate buffered saline solution, water, emulsions, various types of wetting agents, and the like. Suitable carriers comprise those mentioned above and others well known in the art, see, e.g., Remington's Pharmaceutical Sciences, Mack Publishing Company, Easton, Pennsylvania. In an aspect, the pharmaceutical composition can be dissolved in a diluent, prior to administration. The diluent is also selected so as not to affect the biological activity of the botulinum neurotoxin product. Examples of such diluents are distilled water or physiological saline. In addition, the pharmaceutical composition or formulation may also include other carriers or non-toxic, non-therapeutic, non-immunogenic stabilizers and the like. Thus, the formulated botulinum neurotoxin product can be present, in an aspect, in liquid or lyophilized form. In an aspect, it can be present together with glycerol, protein stabilizers (HSA) or non-protein stabilizers such as polyvinyl pyrrolidone (PVP), hyaluronic acid or free amino acids. In an aspect, suitable non-proteinaceous stabilizers are disclosed in WO 2005/007185 or WO 2006/020208. A suitable formulation for HSA-stabilized formulation comprising a botulinum neurotoxin according to the present invention is for example disclosed in U.S. Pat. No. 8,398,998 B2. The formulated botulinum neurotoxin product may be used for human or animal therapy of various diseases or disorders in a therapeutically effective dose or for cosmetic purposes.

### EXAMPLES

General Procedure: A clinical trial was conducted in which 4 consecutive injections were followed by a 16 week observation period each, i.e. 4 consecutive treatment cycles. At the end of each treatment cycle, subjects were examined for eligibility to enter the next cycle. The first treatment cycle (Main Period [MP]) was conducted at two different dose levels of NT 201 (i.e. botulinum toxin serotype A without complexing proteins, Incobotulinumtoxin A) (75 U and 100 U) compared to placebo. Subjects were randomized to the respective treatment with a ratio of 2:2:1 (75 U: 100 U: placebo). The Incobotulinumtoxin A was reconstituted in physiological saline in a concentration of 50 U/mL and the patients received 30 U toxin into each parotid gland and 20 U into each submandibular gland in the 100 U dose group and 22.5 U toxin into each parotid gland and 15 U into each submandibular gland in the 75 U dose group, respectively. In both dose groups the total dose allocated to each parotid and submandibular gland was injected into one site of the respective gland. The MP was followed by 3 consecutive treatment cycles of a dose-blinded extension period where subjects received either 75 U or 100 U NT 201 in the same way as in the MP. Subjects who received placebo during MP were randomized 1:1 to receive either 75 U or 100 U NT 201 during the extension period so the overall dose randomization ratio was 1:1. The results from the MP of the trial show that both the 75 U and 100 U doses reach treatment effects of clinical relevance. They are summarized below.

### Example 1: Results of the Placebo Controlled Main Period (uSFR)

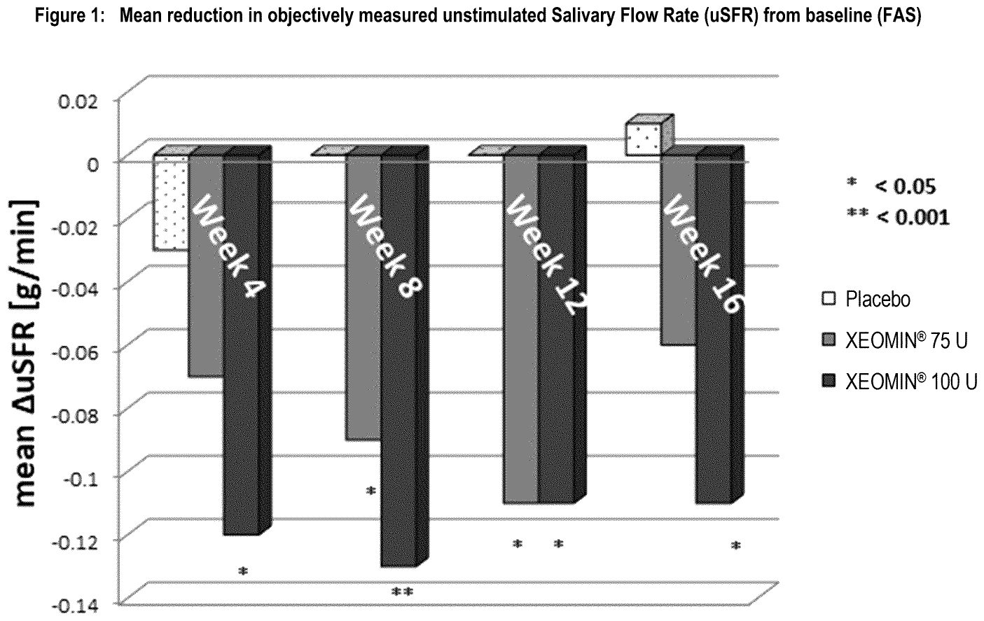

Overall, 184 subjects with chronic troublesome sialorrhea were treated during the MP of the study. The study had two co-primary efficacy endpoints. One of the co-primary efficacy endpoints was the change in the unstimulated salivary flow rate (uSFR) from baseline to week 4 (see Table 1 for mean changes over time). At all time points, the uSFR was meaningfully reduced in both NT 201 treatment groups with the effect being more pronounced in the NT 201 100 U dose group as presented in FIG. 1. At Week 4, statistically significant superiority over placebo was shown for the NT 201 100 U group (p=0.004). Mean uSFR values in the NT 201 75 U with p-values less than 0.05 (Table 1) were reached at Weeks 8 and 12 (p-values: 0.022 and 0.019, respectively). The treatment effects observed in both the NT 201 100 U and NT 201 75 U groups can be considered as clinically relevant.

TABLE 1
Mean uSFR [g/min] at baseline and mean
uSFR changes from baseline over time (FAS)

| | | | P-value | | P-value |
|---|---|---|---|---|---|
| Time | | NT 201 | MMRM* vs. | NT 201 | MMRM* vs. |
| points | Placebo | 75 U | Placebo | 100 U | Placebo |
| Baseline | 0.38 | 0.42 | | 0.40 | |
| Week 4 | −0.03 | −0.07 | 0.542 | −0.12 | 0.004 |
| Week 8 | 0.00 | −0.09 | 0.022 | −0.13 | <0.001 |
| Week 12 | 0.00 | −0.11 | 0.019 | −0.11 | 0.004 |
| Week 16 | 0.01 | −0.06 | 0.180 | −0.11 | 0.002 |

uSFR = unstimulated salivary flow rate [g/min],
FAS = Full Analysis Set,
U = Unit,
MMRM = Mixed Model Repeated Measures
*MMRM uses treatment, country, gender, use of ultrasound and etiology as fixed factors and uSFR at baseline as covariate

### Example 2: Results of the Placebo Controlled Main Period (GICS)

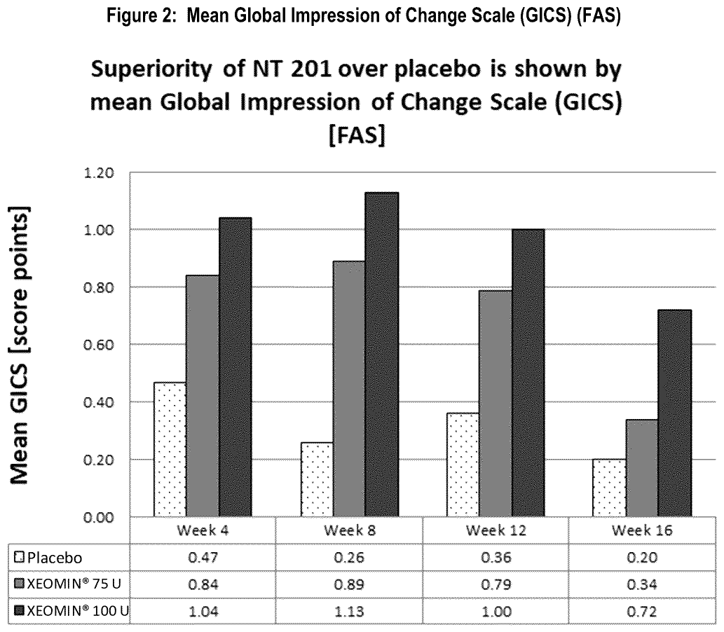

The other co-primary efficacy endpoint was the improvement in global functional scale of subjects measured by the Global Impression of Change Scale (GICS) at Week 4. The GICS is a 7-point Likert scale completed by subjects answering the question “Compared to how you were doing just before the last injection into your salivary gland, what is your overall impression of how you are functioning now as a result of this treatment?” Both dose groups reached an improvement. A statistically significant difference in favor of the 100 U treatment group over the placebo was seen in Week 4 (p=0.002, Table 2, FIG. 2). The 75 U group showed numerically better results compared to placebo at Week 4, but the difference shortly missed statistical significance (p=0.055). Nevertheless, p-values of less than 0.05 were reached in both dose groups at Week 8 and Week 12 and at Week 16 in the 100 U dose group as presented in FIG. 2.

TABLE 2
Mean Subject's GICS values over time (FAS)

| Post | | | P-value | | P-value |
|---|---|---|---|---|---|
| baseline | | NT 201 | MMRM* vs. | NT 201 | MMRM* vs. |
| time point | Placebo | 75 U | Placebo | 100 U | Placebo |
| Week 1 (TC) | +0.47 | +0.54 | 0.689 | +0.76 | 0.065 |
| Week 2 (TC) | +0.63 | +0.72 | 0.626 | +0.91 | 0.096 |
| Week 4 | +0.47 | +0.84 | 0.055 | +1.04 | 0.002 |
| Week 8 | +0.26 | +0.89 | 0.002 | +1.13 | <0.001 |
| Week 12 | +0.36 | +0.79 | 0.035 | +1.00 | 0.001 |
| Week 16 | +0.20 | +0.34 | 0.531 | +0.72 | 0.011 |

GICS = Global Impression of Change Scale,
FAS = Full Analysis Set,
U = Unit,
MMRM = Mixed Model Repeated Measures,
TC = telephone call
*MMRM uses treatment, country, gender, use of ultrasound and etiology as fixed factors and DSFS sum score at baseline as covariate

### Example 3: Results of the Placebo Controlled Main Period (GICS)

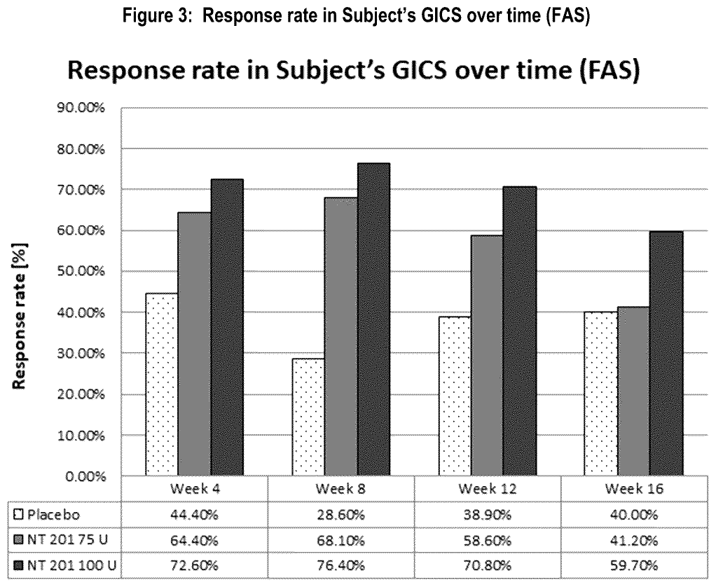

The predefined response criterion for the GICS endpoint to be considered clinically meaningful improvement of drooling was at least one point improvement on the scale (minimally improved). Results of the responder analysis for all treatment groups are presented in Table 3 and FIG. 3.

TABLE 3
Response rate in Subject's GICS (FAS)

| | | | P-value | | P-value |
|---|---|---|---|---|---|
| | | | Fisher's | | Fisher's |
| Post baseline | | NT 201 | exact test | NT 201 | exact test |
| time point | Placebo | 75 U | vs. placebo | 100 U | vs. placebo |
| Week 1 (TC) | 36.1% | 51.4% | 0.157 | 59.5% | 0.026 |
| Week 2 (TC) | 48.6% | 62.2% | 0.215 | 66.2% | 0.095 |
| Week 4 | 44.4% | 64.4% | 0.064 | 72.6% | 0.006 |
| Week 8 | 28.6% | 68.1% | <0.001 | 76.4% | <0.001 |
| Week 12 | 38.9% | 58.6% | 0.066 | 70.8% | 0.002 |
| Week 16 | 40.0% | 41.2% | 1.000 | 59.7% | 0.065 |

FAS = full analysis set,
U = units,
TC = telephone call

The response rate of the placebo group was lower than those of both NT 201 treatment groups throughout the Main Period. It varied from 28.6% (at Week 8) to 48.6% at Week 2. In the two NT 201 groups, the maximal Subject's GICS response rate is reached at Week 8 with 68.1% in the NT 201 75 U group and 76.4% in the NT 201 100 U group. The inventors considers these rates as evidence of clinical meaningfulness of both the NT 201 75 U and 100 U dose groups.

### Example 4: Results of the Placebo Controlled Main Period (DSFS)

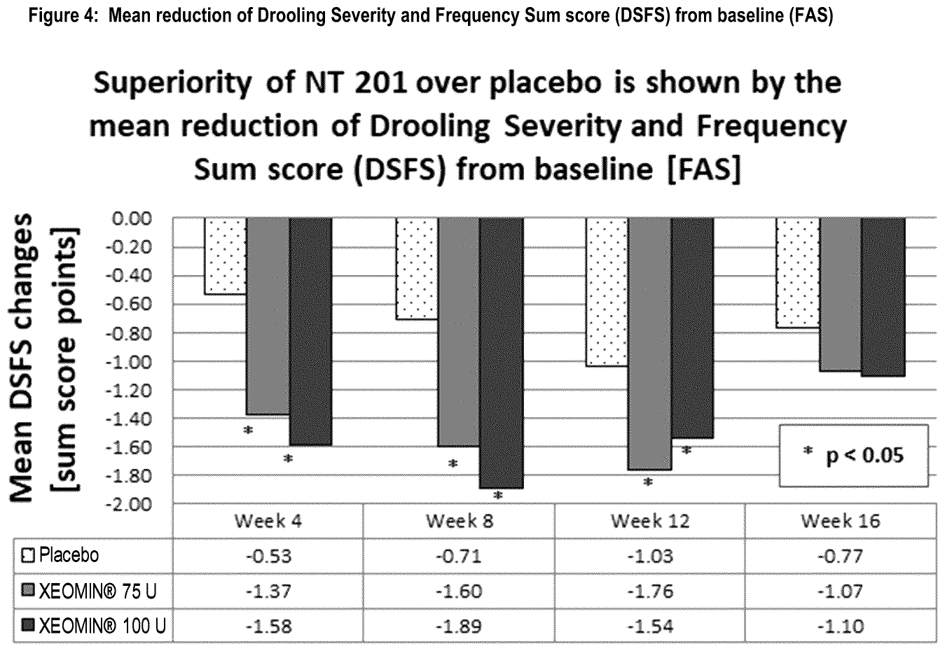

The subjective endpoint Drooling Severity and Frequency Scale (DSFS) was also assessed. The DSFS consists of two sub-scales, a 4-point Likert scale for “drooling frequency” and a 5-point Likert scale for “drooling severity”. Descriptive analyses of DSFS showed clinically relevant improvement of sialorrhea in both NT 201 treatment groups in comparison to no relevant improvement in the placebo group. Mean sum score changes from baseline over time are maximal with an improvement of −1.89 in the 100 U treatment group at Week 8 followed by −1.76 in the 75 U treatment group at Week 12 as presented in Table 4 and FIG. 4. Treatment comparison via Mixed Model Repeated Measures (MMRM) reveals p-values of <0.05 for both NT 201 groups when compared to placebo at Week 4, 8, and 12.

TABLE 4
Mean DSFS sum score at baseline and mean DSFS
sum score changes from baseline over time (FAS)

| | | | P-value | | P-value |
|---|---|---|---|---|---|
| Time | | NT 201 | MMRM* vs. | NT 201 | MMRM* vs. |
| point | Placebo | 75 U | Placebo | 100 U | Placebo |
| Baseline | 6.97 | 6.88 | | 6.78 | |
| Week 4 | −0.53 | −1.35 | 0.002 | −1.55 | <0.001 |
| Week 8 | −0.71 | −1.60 | 0.002 | −1.89 | <0.001 |
| Week 12 | −1.03 | −1.76 | 0.008 | −1.54 | 0.030 |
| Week 16 | −0.77 | −1.07 | 0.223 | −1.10 | 0.116 |

*MMRM uses treatment, country, gender, use of ultrasound and etiology as fixed factors and DSFS sum score at baseline as covariate

### Example 5: Results of the Placebo Controlled Main Period (mROMP)

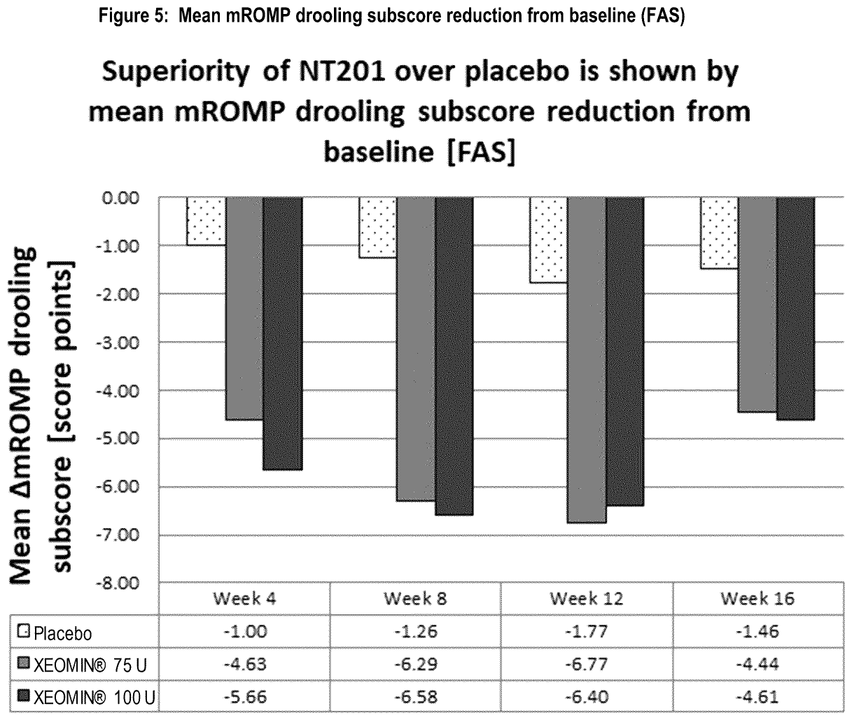

Finally, the modified Radboud Oral Motor Inventory for Parkinson's disease (mROMP) was assessed using the drooling subscale that includes a 9-item questionnaire of 5-point Likert scales. Both NT 201 treatment groups showed superior efficacy results in mROMP drooling in comparison to the placebo group presented in Table 5 and FIG. 5. Mean changes from baseline over time reach a maximum improvement of −6.58 in the 100 U treatment group at Week 8 and −6.77 in the 75 U treatment group at Week 12. The inventors conclude that treatment effects seen in both dose groups were superior over the effect of placebo and NT 201 effects were consistent among all measures and robust throughout the observation to confirm appropriate clinical relevance of both doses.

TABLE 5
Change in mROMP drooling scores from study baseline
to weeks 4, 8, 12 and 16-MP (FAS, OC)

| Placebo | | NT 201 75 U | | NT 201 100 U | |
|---|---|---|---|---|---|
| (N = 36) | | (N = 74) | | (N = 74) | |
| n obs | Mean (SD) | n obs | Mean (SD) | n obs | Mean (SD) |

Change from study baseline

| | | | | | | |
|---|---|---|---|---|---|---|
| Week 4 | 36 | −1.00 (4.71) | 72 | −4.63 (5.26) | 73 | −5.66 (6.16) |
| Week 8 | 35 | −1.26 (4.91) | 72 | −6.29 (6.52) | 72 | −6.58 (5.90) |
| Week 12 | 35 | −1.77 (4.54) | 70 | −6.77 (6.05) | 72 | −6.40 (5.20) |
| Week 16 | 35 | −1.46 (5.03) | 68 | −4.44 (5.56) | 70 | −4.61 (5.40) |

Score ranges from 9 (best) to 45 (worst).

### Example 6: Subgroup Analysis of Results of the Placebo Controlled Main Period (mROMP)

Subgroup analysis by etiology showed that subjects with sialorrhea after stroke in the NT 201 100 U group had higher mean decreases in uSFR than subjects with sialorrhea associated with Parkinson's disease or atypical parkinsonism (Table 6).

TABLE 6
Subgroup analysis of change in uSFR from study baseline
to week 4-MP (Full Analysis Set FAS, Observed Cases OC)

| | Placebo | | NT 201 75 U | | NT 201 100 U | |
|---|---|---|---|---|---|---|
| Etiology of sialorrhea | n obs | Mean (SD) | n obs | Mean (SD) | n obs | Mean (SD) |
| Sialorrhea associated | 29 | −0.04 (0.23) | 58 | −0.08 (0.15) | 58 | −0.11 (0.19) |
| with Parkinson's disease | | | | | | |
| or atypical parkinsonism | | | | | | |
| Sialorrhea after stroke | 6 | 0.04 (0.12) | 13 | −0.02 (0.14) | 13 | −0.20 (0.28) |
| Sialorrhea after TBI | 1 | −0.02 (—) | 2 | −0.07 (0.37) | 2 | −0.12 (0.10) |

uSFR is given in g/min
DSFS sum score ranges from 2 (best) to 9 (worst).

### Example 7: Efficacy in 3 Consecutive Treatment Cycles with 16 Weeks Intervals

Efficacy results in 3 consecutive treatment cycles with 16 weeks intervals provided evidence for further improvement of sialorrhea. The change in uSFR from study baseline to all observation time points after the second injection, and the change from each injection (weeks 16, 32 and 48 after the first injection) to the respective assessment time points (weeks 20, 36 and 52 after the first injection), and to the end-of-cycle/end-of-study visits (weeks 32, 48 and 64 after the first injection) in each cycle was evaluated also.

Summary statistics for the uSFR at the cycle baselines of the extension period (EP) without placebo control group are displayed in Table 7. (Subjects randomized to the placebo group at the MP were randomized within the same setting to the 75 U or 100 U dose group in a 1:1 randomization ratio for the EP. Subjects in the 75 U or 100 U dose group in MP were maintained on their dose in the EP). The mean uSFR in the EP in both NT 201 treatment groups was highest at the cycle 2 baseline and lowest at the cycle 4 baseline. Additionally, the mean uSFR at each cycle baseline were slightly higher in the NT 201 75 U group than in the NT 201 100 U group. Similar improvement of sialorrhea was observed when GICS, DSFS and mROMP were analyzed for NT201 100 U and NT201 75 U over the extension period.

TABLE 7
Mean uSFR at all cycle baselines - EP (Safety
Evaluation Set SES-EP, Observed Cases OC)

| | NT 201 75 U | | NT 201 100 U | |
|---|---|---|---|---|
| | n obs | Mean (SD) | n obs | Mean (SD) |
| Cycle 2 Baseline | 83 | 0.38 (0.25) | 89 | 0.30 (0.18) |
| Cycle 3 Baseline | 79 | 0.31 (0.22) | 84 | 0.24 (0.17) |
| Cycle 4 Baseline | 79 | 0.28 (0.24) | 78 | 0.23 (0.16) |

uSFR is given in g/min.
Randomized treatment group was used.

TABLE 8
Dosing table for botulinum toxin administration into
parotid and submandibular glands in children.

| | Parotid gland, | | Submandibular gland, | | | |
|---|---|---|---|---|---|---|
| | each side | | each side | | | Total |
| Body | Total dose | Volume per | Total dose | Volume per | Total dose | injection |
| weight | per gland | injection | per gland | injection | (both sides) | volume |
| [kg] | [units] | [ml] | [units] | [ml] | [units] | [ml] |
| ≥12 <15 | 6 | 0.24 | 4 | 0.16 | 20 | 0.8 |
| ≥15 <19 | 9 | 0.36 | 6 | 0.24 | 30 | 1.2 |
| ≥19 <23 | 12 | 0.48 | 8 | 0.32 | 40 | 1.6 |
| ≥23 <27 | 15 | 0.60 | 10 | 0.40 | 50 | 2.0 |
| ≥27 <30 | 18 | 0.72 | 12 | 0.48 | 60 | 2.4 |
| ≥30 | 22.5 | 0.90 | 15 | 0.60 | 75 | 3.0 |

1. A method for treating sialorrhea or increased saliva production in a patient, the method comprising administering a therapeutically effective amount of a botulinum neurotoxin by injection into the parotid glands and submandibular glands of the patient, in at least 3 consecutive treatment cycles,
wherein the ratio between the amount of the botulinum neurotoxin administered into each of the parotid glands and each of the submandibular glands is between 1.45 to 1 and 1.7 to 1,
wherein there is a time interval between consecutive treatment cycles of between 14 and 18 weeks,
and wherein the botulinum neurotoxin is injected into one site of each of the submandibular glands and into one site of each of the parotid glands.

2. The method according to claim 1, wherein the total dose of said botulinum neurotoxin administered into the parotid glands and submandibular glands is between 70 U and 110 U.

3. The method according to claim 1, wherein said botulinum neurotoxin is administered in an aqueous composition having a botulinum neurotoxin concentration in the range between 45 and 55 U/mL.

4. The method according to claim 1, wherein said botulinum neurotoxin is administered in 0.3 to 0.5 ml per injection site into the submandibular glands and in 0.5 to 0.7 ml per injection site into the parotid glands.

5. The method according to claim 1, wherein the botulinum neurotoxin is injected into the parotid glands and submandibular glands using ultrasound guidance.

6. The method according to claim 1, wherein said botulinum neurotoxin is a botulinum neurotoxin complex.

7. The method according to claim 1, wherein said botulinum neurotoxin is the neurotoxic component of a botulinum neurotoxin complex, wherein said neurotoxic component is devoid of any other protein component of the Clostridium botulinum neurotoxin complex.

8. The method according to claim 1, wherein said botulinum neurotoxin is selected from the group of botulinum neurotoxin serotypes, consisting of botulinum neurotoxin serotype A, botulinum neurotoxin serotype B, botulinum neurotoxin serotype Cl, botulinum neurotoxin serotype D, botulinum neurotoxin serotype E, botulinum neurotoxin serotype F and botulinum neurotoxin serotype G.

9. The method according to claim 1, wherein the sialorrhea or increased saliva production is further associated with a disease or condition selected from the group consisting of Amyotrophic lateral sclerosis (ALS), Progressive Supranuclear Palsy, Corticobasal Degeneration, Multiple System Atrophy, stroke, traumatic brain injury (TBI), clozapine induced hypersalivation, Rett syndrome, Angelman syndrome, epileptic encephalopathy, brain tumours, total pharyngolaryngectomy, supracricoid laryngectomy, supraglottic laryngectomy, dementia, and intellectual disability.

10. The method according to claim 9, wherein the disease or condition is associated with stroke.

11. The method according to claim 1, wherein the time interval is between 15 and 17 weeks.

12. The method according to claim 11, wherein the time interval is 16 weeks.


SEQUENCE LISTING (14 sequences)

SEQ ID NO: 1 - DNA, 3891 bp, Clostridium botulinum
  source 1..3891: mol_type = other DNA
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3876 bp, Clostridium botulinum
  source 1..3876: mol_type = other DNA
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 4 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 3843 bp, Clostridium botulinum
  source 1..3843: mol_type = other DNA
atgccaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctcctcgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataaggtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgccataaagcaatagatggtagatcattatataataaaacattagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatataattaatgaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatacaataattaatagtataaaacaaaattctgggtggaaattatgtattaggaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttaaatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatattattaatgataattatatagataggtatatagcacctaaaagtaatatacttgtacttgttcagtatccagatagatctaaattatatactggaaatcctattactattaaatcagtatctgataagaatccttatagtagaattttaaatggagataatataatgtttcatatgttatataatagtgggaaatatatgataataagagatactgatacaatatatgcaatagaaggaagagagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagtaa

SEQ ID NO: 6 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 7 - DNA, 3858 bp, Clostridium botulinum
  source 1..3858: mol_type = other DNA
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcctagacctacttcaaagtatcaaagttattatgatcctagttatttatctactgatgagcaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattgtatggaataaatataccatctgataaaaggattcgtccacaagttagcgagggatttttttctcaagatggacccaacgtacaatttgaggaattatacacatttggaggatcagatgttgaaataatacctcaaattgaaagattacaattaagagaaaaagcattaggtcactataaagatatagcgaaaagacttaataatattaataaaactattccttctagttggagtagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaagcattatctacctgtatttgcaaatatattagatgataatatttatactataataaacggttttaatttaacaactaaaggttttaatatagaaaattcgggtcagaatatagaaaggaatcctgcactacaaaaacttagttcagaaagtgtagtagatttgtttacaaaagtatgtttaagattaacaagaaatagtagagatgattcaacatgtattcaagttaaaaataatacattaccttatgtagctgataaagatagcatttcacaagaaatatttgaaagtcaaattattacagatgagactaatgtagaaaattattcagataatttttcattagatgaatctattttagatgcaaaagtccctactaatcctgaagcagtagatccactgttacccaatgttaatatggaacctttaaatgttccaggtgaagaagaagtattttatgatgatattactaaagatgttgattatttaaactcttattattatttggaagcccaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaaaaaagatacattggataaaatatcagatgtatcagccataattccatatataggacctgccttaaatataggaaattcagcattaaggggaaactttaagcaagcatttgcaacagctggtgtagcttttttgttagaaggatttccagagtttacaatacctgcactcggtgtatttaccttttatagttctattcaagaaagagagaaaattattaaaactatagaaaattgtttagaacaaagagttaagagatggaaagattcatatcaatggatggtatcaaattggttgtcaagaattactactcgatttaatcatataagttatcaaatgtatgattctttgagttatcaggcagatgcaatcaaagctaaaatagatttagaatataaaaaatactcaggaagtgataaagaaaatataaaaagtcaagttgaaaatttaaaaaatagtttagatgtaaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatatgattaatgaatatttcaatagtattaatgattcaaaaattttgagcttacaaaataaaaaaaatactttgatggatacatcaggatataacgcagaagtgagagtagaaggcaatgttcagcttaatccaatatttccatttgactttaaattaggtagttcaggggatgatagaggtaaagttatagtaacccagaatgaaaatattgtatataatgctatgtatgaaagttttagtattagtttttggattaggataaataaatgggtaagtaatttacctggatatactataattgatagtgttaaaaataactcaggttggagtataggtattattagtaattttttagtgtttactttaaaacaaaatgaaaatagtgaacaagatataaactttagttatgatatatcaaagaatgctgcgggatataataaatggttttttgtaactattactaccaatatgatgggaaatatgatgatttatataaatggaaaattaatagatactataaaagttaaagagttaactggaattaattttagcaaaactataacatttcaaatgaataaaattccaaatactggcttaattacctcagattctgataacatcaatatgtggataagggatttttatatctttgctaaagaattagatgataaagatattaatatattatttaatagcttgcaatatactaatgttgtaaaagattattggggaaatgatttaagatatgataaagaatattacatgattaacgtaaattatatgaatagatatatgtctaaaaaaggcaatggaattgtttttaatacacgtaaaaataataatgacttcaatgaaggatataaaattataataaaaagaattagaggaaatacaaatgatactagagtacgaggagaaaatgtattatattttaatactacaattgataacaaacaatatagtttaggtatgtataaaccttctagaaatctagggactgatttagttccactaggtgcattggatcaaccaatggatgagatacgtaaatatggttcgtttataatacaaccatgcaatacttttgattactatgcatcacaattatttttgtcaagtaatgcaacaacaaataggcttggaatactatcaattggtagttatagtttcaaacttggagatgactattggtttaatcacgaatatttaattcctgttataaaaatagagcattatgcttcattattagaatcaacatcaactcattgggtttttgtacctgcaagtgaataa

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 9 - DNA, 3756 bp, Clostridium botulinum
  source 1..3756: mol_type = other DNA
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - DNA, 3843 bp, Clostridium botulinum
  source 1..3843: mol_type = other DNA
atgccagttgtaataaatagttttaattataatgaccctgttaatgatgagacaattttatacatgcagaaaccatatgaagaaagaagtagaaaatattataaagcttttgagattatgcctaatgtttggataatgcctgagagagatacaataggaactaagcctgatgagtttcaggtgccggattcattaaagaacggaagtagtgcttattatgatcctaattatttaaccactgatgctgaaaaagatagatatttaaaaacaatgataaaattatttaatagaattaatagtaatcctacagggaaagttttgttagaagaagtatcaaatgctagaccatatttaggagatgatgacacgctaattaatgaattccttccagttaatgtaactacaagtgttaatataaaattttcaactgatgttgaaagttcaataatatcgaatcttcttgtattgggagcaggacctgatatatttaaagcttactgtaccccccttgtaaggtttaataagtcagataaattaattgaaccaagtaatcatggttttggatcaattaatatcttgacattttcacctgagtatgaacatatttttaatgatattagtggagggaatcataatagtacagaatcatttattgcagatcctgcaatttcactagctcatgaattgatacatgcactacatggattatacggggctaaggcagttactcataaagagtctctagtagcagagcgaggacctcttatgatagccgaaaagcccataaggctagaagaatttttaacttttggaggtgaggatttaaatatcattcctagtgctatgaaggaaaaaatatataacgatcttttagctaactatgaaaaaatagctactagacttagagaagttaatacggctcctcctggatatgatattaatgaatataaagattattttcaatggaagtatggactagatagaaatgcagatggaagttatactgtgaatagaaataaatttaatgaaatttataaaaaattatatagctttacagagattgacttagcaaataaatttaaagtaaaatgtagaaatacttattttattaaatatggatttgtaaaagttccaaatttgttagatgatgatatttatactgtatcagaggggtttaatataggtaatttagcagtaaacaatcgcggacaaaatataaatttaaatcctaaaattattgattccattccagataaaggtttagtggaaaagattattaaattttgtaagagcattattcctagaaaaggtacgaagcagtcaccgtcactatgcattagagtaaataatagggagttattttttgtagcttcagaaagtagctataatgaaagtgatattaatacacctaaagaaattgacgatacaacaaatctaaataataattatagaaataatttagatgaagttattttagattataatagtgagacaatacctcaaatatcaaatcgaacattaaatacacttgtacaagacaatagttatgtgccaagatatgattctaatggaacaagtgaaatagaggaatatgatgttgttgactttaatgtatttttctatttacatgcacaaaaagtaccagaaggtgaaaccaatataagtttaacttcttcaattgatacagcattattagaagaatccaaagtatatacatttttttcttcagagtttatcgatactatcaataaacctgtaaatgcagcactatttatagattggataagcaaagtaataagagattttaccactgaagctacacaaaaaagtactgttgataagattgcagacatatctttaattgtaccctatgtaggtcttgctttgaatatagttattgaggcagaaaaaggaaattttgaggaggcatttgaattattaggagcgggtattttattagaatttgtgccagagcttacaattcctgtaattttagtgtttacgataaaatcctatatagattcatatgagaataaaaataaagcaattaaagcaataaataattcattaatcgaaagagaagcaaagtggaaagaaatatatagttggatagtatcaaattggcttactagaattaatacgcaatttaataaaagaaaagagcaaatgtatcaggctttacaaaatcaagtagatgcaataaaaacagcaatagaatataaatataataattatacttcagatgagaaaaatagacttgaatctaaatataatatcaataatatagaagaagaattgaataaaaaagtttctttagcaatgaaaaatatagaaagatttatgacagaaagttctatatcttatttaatgaaattaataaatgaagccgaagttggtaaattaaaagaatatgataaacatgttaagagcgatttattagactatattctctaccataaattaatcttaggagagcagacaaaggaattaattgatttggtgactagtactttgaatagtagtattccatttgaactttcttcatatactaatgataaaattctaattatatattttaatagattatataaaaaaattaaagatagttctattttagatatgcgatatgaaaataataaatttatagatatctctggatatggttcaaatataagcattaatggaaacgtatatatttattcaacaaatagaaatcaatttggaatatatagtggtaggcttagtgaagttaatatagctcaaaataatgatattatatacaatagtagatatcaaaattttagtattagtttctgggtaaccattcctaaacactacagacctatgaatcgtaatcgggaatacactataataaattgtatggggaataataattcgggatggaaaatatcacttagaactattagagattgtgaaataatttggactttacaagatacttccggaaataaggaaaaattaatttttaggtatgaagaacttgctagtatatctgattatataaataaatggatttttgtaactattactaataatagattaggcaattctagaatttacatcaatggaaatttaatagttgaaaaatcaatttcgaatttaggtgatattcatgttagtgataatatattatttaaaattgttggttgtgatgatgaaacgtatgttggtataagatattttaaagtttttaatacggaattagataaaacagaaattgagactttatatagtaatgagccagatccaagtatcttaaaagactattggggaaattatttgctatataataaaaaatattatttattcaatttactaagaaaagataagtatattactcggaattcaggcattttaaatattaatcaacaaagaggtgttactggaggcatatctgtttttttgaactataaattatatgaaggagtagaagttattataagaaaaaatgctcctatagatatatctaatacagataattttgttagaaaaaacgatctagcatacattaatgtagtagatcatggtgtagaatatcggttatatgctgatatatcaattacaaaatcagagaaaataataaaattaataagaacatctaatccaaacgatagcttaggtcaaattatagttatggattcaataggaaataattgcacaatgaattttcaaaacaatgatgggagcaatataggattactaggttttcattcagatgatttggttgctagtagttggtattataaccatatacgaagaaacactagcagtaatggatgcttttggagttttatttctaaagagcatggttggaaagaataa

SEQ ID NO: 12 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 13 - DNA, 3894 bp, Clostridium botulinum
  variation 20: replace = n
  source 1..3894: mol_type = other DNA
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 14 - protein, 1297 aa, Clostridium botulinum
  UNSURE 7: note = Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE